(19) United States
(12) Patent Application Publication
(10) Pub. No.: US 2001/0012916 A1
(43) Pub. Date: Aug. 9, 2001

(54) Blood pressure measuring device

(76) Inventor: Klaus Deuter, Unterschleissheim (DE)

Correspondence Address: McCormick, Paulding & Huber, City Place II, 185 Asylum Street, Hartford, CT, 06103-3402, US

(21) Appl. No.: 09/746,425

(22) Filed: Dec. 21, 2000

(30) Foreign Application Priority Data
Dec. 23, 1999 (DE) 199 62 700.2

(51) Int. Cl. A61B 5/02

(52) U.S. Cl. 600/485; 600/500; 600/494; 600/503

(57) ABSTRACT

9A blood pressure measuring device has a pressure cuff the inflation of which is automatically effected and controlled by an evaluation and control unit to allow a continuous monitoring of a patient's blood pressure, especially during the night without infringing on the patient's comfort and sleep. The device also includes an EKG device and a sensor for sensing the pressure in the cuff. In a first mode of operation, the control and evaluation unit calculates an estimated blood pressure value from pulse wave transmission times with each calculated pulse wave transmission time being the time elapsing between a heart beat as detected by the EKG device and a corresponding cuff pressure change detected by the pressure sensor. In the event of the appearance of blood pressure spikes while operating in the first mode, the device switches to a second operating mode during which absolute blood pressure values are determined.

### FIELD OF THE INVENTION

The invention concerns a blood pressure measuring device including an inflatable pressure cuff which is applicable to a body extremity and which is connected with an automatically operating pressurized air source and with a pressure sensor for sensing the cuff pressure and also having a control and evaluation unit connected with the pressurized air source, the pressure sensor and a controllable valve of the pressure cuff.

### BACKGROUND OF THE INVENTION

With certain patients, such as for example diabetics, it can be necessary to supervise the blood pressure continuously during the night. It is known, that the appearance of kidney damage in diabetics is decisively dependent on the blood pressure level. The appearance of blood pressure spikes can be monitored only very incompletely with customary 24-hour blood pressure monitors because of a measurement being taken mostly only every 30 minutes because of the great encroachment on the patient during the night. The blood pressure spikes which appear are, however, largely much shorter and, therefore, fall many times in the gaps between the measurements. Moreover, known 24-hour blood pressure monitors are relatively expensive and, therefore, are unsuited for wide use as home devices.

The object of the invention is basically to provide a blood pressure measuring device which is economical and of simple construction and which allows for a continuous monitoring of the blood pressure of a patient with little disturbance to the patient.

### SUMMARY OF THE INVENTION

This object is solved by a blood pressure measuring device of the above-mentioned kind wherein the cuff pressure is adjustable and controllable to a pregiven value and wherein the control and evaluation unit is connected to an EKG-measuring apparatus having at least two EKG electrodes and which is so formed that a pulse wave transmission time is determined from an EKG-signal produced by the EKG measuring device and from a pressure signal of the pressure sensor.

The invention rests on the recognition that a monitoring of the blood pressure should take place continuously but that a determination of the precise blood pressure value is of less importance. For the doctor it is of primary interest whether blood pressure spikes appear and approximately how high the spikes are.

One such measurement can be made according to a method known in itself by measurement of the pulse wave transmission time. In this method, the time interval between the heart beat (sensed by an EKG) and the arrival of the heart beat pulse wave at an extremity such as the wrist, and sensed by a pressure sensor, is determined. This time interval is specific to the patient and dependent on the blood pressure. Therefore, a calibration measurement must be carried out with a blood pressure measuring device measuring the absolute pressure. Moreover, the systolic pressure and the diastolic pressure cannot be separately sensed and therefore an average value is determined.

The greatest problem of the known method for determining the pulse wave transmission time previously resided in that an interference free simple derivation of the pulse could not be realized over long time periods with simple means. The sensing with an infrared-plethysmograph sensor as well as with an ultrasonic sensor is very unreliable, since a sensor has to be constantly precisely positioned. This is extremely problematic at a spot on the patient such as the wrist, especially when this is uncontrollably moved during sleep.

The device of the invention can be realized in the form of a device carried by a wrist which is connected by a thin cable to an EKG adhesive electrode placed on the patient's chest. Thereby, together with an opposite electrode on the pressure cuff, a simple EKG derivation can be realized. This device can without large disturbance to the patient be easily worn on the wrist during the night. In contrast to the absolute measurement of the blood pressure, where for the measurement of the systolic pressure, the blood circulation has to be entirely interrupted by the cuff pressure, in the device of the invention, the cuff pressure is only adjusted to a value which is slightly above the diastolic blood pressure so that pulse waves which pass through the cuff create pressure impulses which are measurable. This moderate pressure of the pressure cuff barely disturbs the patient.

Preferably the blood pressure measuring device of the invention is switchable between a first operating mode for the determination of the pulse wave transmission time and a second operating mode for the absolute measurement of the blood pressure by means of the pressure cuff. Therefore, the blood pressure measuring device on one hand can be used for continuous monitoring by means of the determination of the pulse wave transmission time and on the other hand can be used for a precise measurement of the blood pressure. For this purpose, the control and evaluation unit advantageously has means for correlating the blood pressure values obtained by measurements of the absolute blood pressure to the blood pressure values determined on the basis of the pulse wave transmission times, so that in this way a calibration of the pulse wave transmission time blood pressure values for the individual patients can be made.

In a preferred embodiment of the invention, the control and evaluation unit is so formed that it, in dependence on pregiven conditions, switches from the first operating mode to the second operating mode. One such condition can be the appearance of pressure spikes. If during the first operating mode, it is determined that blood pressure spikes appear, the device is switched over to the second operating mode and carries out a precise blood pressure measurement.

Advantageously, the control and evaluation unit is so formed that the cuff pressure prevailing during the first operating mode is adjustable in dependence on the blood pressure values determined during the second operating mode. Otherwise, it may happen that the course of the pulse waves cannot be sensed because the diastolic blood pressure has, for example, risen above the set pressure of the cuff.

For the same reason, it can also be advantageous to provide that the cuff pressure prevailing during the first operating mode is changeable in dependence on the amplitude of the pressure signals sensed during the first operating mode. If it happens that this amplitude is too small and threatens to disappear entirely, the cuff pressure is slightly increased. On the other hand, it can be lowered if the amplitude values are too high, in order to subject the patient as little as possible to a tightly pumped up pressure cuff.

In a special embodiment of the inventive blood pressure measuring device, the control and evaluation unit is so formed that it determines a characteristic value for the blood pressure from the cuff pressure prevailing during the first operating mode and the amplitude of pressure signals sensed in this first operating mode. This can take place without the aid of the pulse wave transmission time.

The control and evaluation unit is advantageously connected to an indicator device in order to facilitate the operation of the device for the carrier of the device or the doctor and to indicate the measured values. It can also be connected with an alarm apparatus in order to make the patient aware of critical conditions arising in the nighttime.

For the continuous monitoring of the blood pressure, it is advantageous if the control and evaluation unit has a memory for storing measured data from a plurality of measuring cycles. This data can then, for example, be transmitted through an interface to an external data processing and/or output apparatus.

As has already been mentioned above, advantageously one of the EKG-electrodes is arranged directly on the pressure cuff, which in turn can also be conducting and therefore formed as the EKG-electrode. In place of an adhesive electrode intended for adhesive attachment to the patient's chest, a contact surface can also be provided on the measuring device which the patient touches with a finger of one hand. In this way, the measuring apparatus of the invention can also be used as a mobile pulse wave transmission time measuring device or as an emergency EKG measuring device.

### BRIEF DESCRIPTION OF THE DRAWINGS

Further features and advantages of the invention will be apparent from the following description which, in connection with the accompanying drawings, explains the invention by way of an exemplary embodiment. The drawings are:

FIG. 1—A schematic perspective view of a blood pressure measuring device of the invention which is to be applied to the wrist of a patient, and

FIG. 2—A schematic view of the blood pressure measuring device wherein the individual components of the measuring device are represented in the form of a block diagram.

### DETAILED DESCRIPTION OF PREFERRED EMBODIMENT

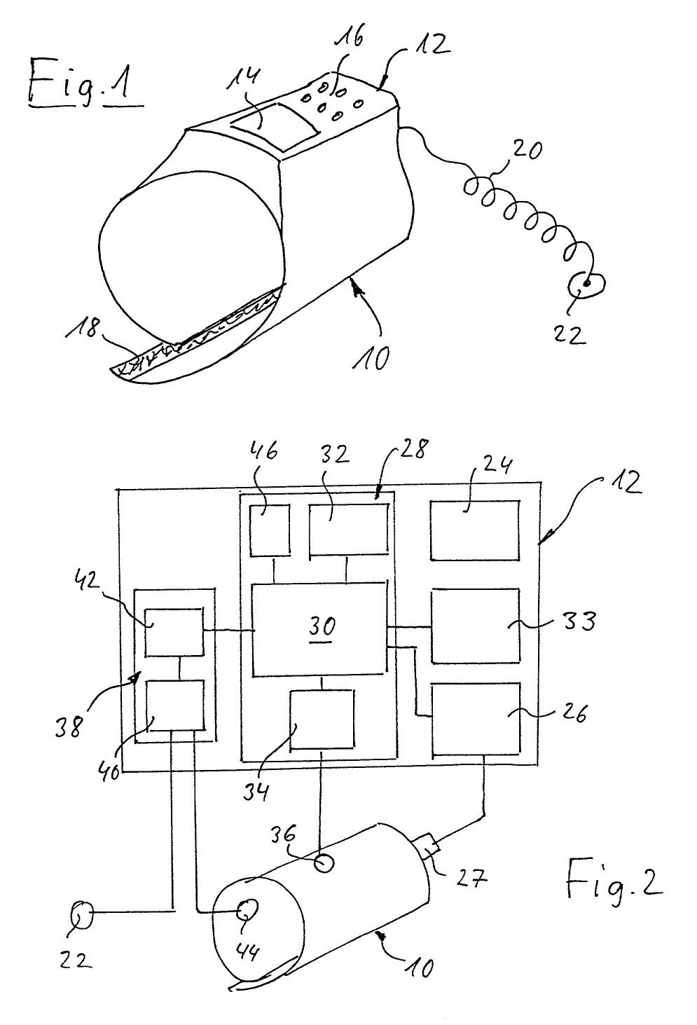

The blood pressure measuring device according to the invention and illustrated in FIG. 1 includes a pressure cuff 10 and a measuring device housing 12 connected with the cuff and having an indicator unit 14 and an operating field 16. The pressure cuff 10 is of such size that it can be applied to the wrist of a patient with it being closable in a known way as, for example, by means of a Velcro fastener 18. An EKG-adhesive electrode 22 is connected with the measuring device housing 12 by a thin cable 20 so that the cable can be, for example, led along the arm to the breast of the patient without the patient being substantially hindered in his movement by the applied measuring device.

The structure of the measuring device can be understood in detail from FIG. 2.

Inside of the measuring device housing 12 is a current source 24 which is connected in a non-illustrated way with the individual components of the measuring device and which, for example, can be formed by a battery. An electrically driven pump 26 is connected to the pressure cuff 10 through a controllable valve 27 for the purpose of inflating the cuff and maintaining a predetermined pressure inside of the cuff. A control and evaluation unit 28 includes a microprocessor 30, which carries out all of the control and evaluation functions of the measuring device and which is connected with a data memory 32 for storing the measured data. The processor 30 is further connected with an indicator/alarm device 33 and with the pump 26. Also, a pressure sensor 36 arranged on the cuff 10 is connected with the processor 30 through a converter 34 arranged on the cuff 10.

Inside of the measuring device housing 12 is also an EKG-measuring unit 38 with an amplifier 40 and an analogue to digital converter 42 which unit 38 is connected with the adhesive electrode 22 and a second EKG electrode 44 arranged on the cuff 10. The analogue to digital converter 42 is connected with the processor 30.

The control and evaluation unit 28 can be connected to a non-illustrated external data processing and/or output device through an interface 46.

The so far described blood pressure measuring device operates in the following way. After the fastening of the pressure cuff 10 to the wrist of the patient or carrier, an operating mode is first switched to whereby in a customary way, e.g., according to the oscillometric method, the blood pressure, that is the systolic and the diastolic blood pressure, is measured. Then a switch is made to another operating mode in which the pulse wave transmission time is determined. This takes place by measurement of the time between an EKG-signal obtained by means of the electrodes 22, 44 and from which through a beat recognizing algorithm the moment of the heart muscle contraction is determined, and the moment the related pulse wave signal is captured by the pressure sensor 36. Preferably these measurements are repeated a number of times alternately with different absolute blood pressure values. By the correlation of the measured pulse wave transmission times with the associated blood pressure values, a characteristic curve is established which permits an estimation of the blood pressure on the basis of the pulse wave transmission time measurements.

After the establishment of the characteristic curve, the device automatically switches to the operating mode for a measurement of the pulse wave transmission time. For that, the pressure cuff 10 by means of the pump 26 is pumped up to a pressure value slightly above the diastolic pressure, so that pressure impulses in the pressure cuff 10 arising from the pulse waves can be captured by the pressure sensor 36. At the same time, the measuring device begins to sense the heart beat complex by means of the EKG-measuring device 38 and to measure the pulse wave transmission times in milliseconds. The values measured at regular intervals are then evaluated with reference to the established characteristic curve so that a blood pressure estimation can continuously be made. The measured pulse wave transmission time and, as the case may be, the estimated blood pressure values are stored for later evaluation.

A supervisory algorithm in the control and evaluation unit 28 recognizes when the estimated blood pressure value exceeds a pregiven threshold value. In this case, the device switches to the mode for an absolute measurement of the blood pressure and carries out one or more such blood pressure measurements. The corresponding results are likewise stored. When the upper blood pressure threshold value is again not exceeded, the device switches automatically back to the pulse wave transmission time mode.

After the end of the measuring time, the measured results are transmitted from the memory 32 through the interface 46 for analysis either to a PC or another device, so that they can be evaluated by a doctor.

With the previously described device, the doctor obtains a gapless record of the blood pressure course. Therefore, the blood pressure adjustment of a patient can be much better assessed than it can with customary 24-hour devices. The patient's stress is substantially lower in comparison to customary devices since in the case of normal blood pressure course, essentially no absolute value measurements need be carried out with accompanying high cuff pressure. The patient can apply the device very easily by himself. The device of the invention, in comparison to customary 24-hour devices, can be made at less cost and for patients at risk can be used for a constant monitoring at home. As the case may be, the data produced by the device can also be transmitted on line to a clinical center and can there be evaluated.

The alarm function of the measuring device can be used to warn the patient upon the appearance of blood pressure spikes and, for example, to invite the taking of medicine. The device can also be used as a simple economical pulse wave transmission time measuring device. In this case, the EKG adhesive electrode 22 can be replaced by a contact surface on the measuring device itself, which contact surface can be touched by a finger of the other hand and thereby enable an EKG determination. One such device would be ideal for the emergency bag of an emergency doctor. As the case may be, the EKG-measuring unit 38 can also be so formed that it is connectable with a recording device so that the doctor during an occurring blood pressure crisis also has the EKG available for analysis.

1. A blood pressure measuring device comprising:
an inflatable pressure cuff (10) applicable to a body extremity and connected with an automatically operating pressurized air source (26) and a pressure sensor (36) for sensing the cuff pressure, and a control and evaluating unit (28) connected with the pressurized air source (26), the pressure sensor (36) and a controllable valve (27) of the pressure cuff (10), wherein the cuff pressure is adjustable and controllable to a pregiven value and the control and evaluation unit (28) is connected with an EKG-measuring apparatus (38) having at least two EKG-electrodes (22, 44), with the control and evaluation unit (28) determining the pulse wave transmission time from the EKG-signal produced by the EKG measuring device (38) and from a pressure signal produced by the pressure sensor (36).

2. A blood pressure measuring device according to claim 1 wherein said blood pressure measuring device is switchable between a first operating mode for determining the pulse wave transmission time and a second operating mode for making an absolute measurement of the blood pressure by means of said pressure cuff (10).

3. A blood pressure measuring device according to claim 1 wherein the control and evaluation unit (28) has means for correlating blood pressure values obtained by the absolute measurements to blood pressure values determined on the basis of pulse wave transmission times.

4. A blood pressure measuring device according to claim 2 wherein the control and evaluation unit (28) is so designed that it, in dependence on pregiven conditions switches out of the first operating mode into the second operating mode.

5. A blood pressure measuring device according to claim 2 wherein the cuff pressure prevailing during the first operating mode is adjustable in dependence on the blood pressure values sensed during the second operating mode.

6. A blood pressure measuring device according to claim 2 wherein the cuff pressure prevailing during the first operating mode is variable in dependence on the amplitude of pressure pulsation signals obtained during the first operating mode.

7. A blood pressure measuring device according to claim 2 wherein the control and evaluation unit (28) is so designed that it determines a characteristic magnitude for the blood pressure from the cuff pressure prevailing during the first operating mode and the amplitude of the pressure pulsation signals obtained in the first operating mode.

8. A blood pressure measuring device according to claim 1 wherein the control and evaluation unit (28) is connected with an indicator device (33).

9. A blood pressure measuring device according to claim 1 wherein the control and evaluation unit (28) is connected with an alarm device (33).

10. A blood pressure measuring device according to claim 1 wherein the control and evaluation unit (28) has a memory (32) for storing the measured data obtained over a plurality of measuring cycles.

11. A blood pressure measuring device according to claim 1 wherein the control and evaluation unit (28) has an interface (46) for connection to an external data processing device and/or data output device.

12. A blood pressure measuring device according to claim 1 wherein one of the EKG electrodes (44) is so arranged on the pressure cuff (10) that it engages the body extremity enclosed by the pressure cuff (10).

13. A blood pressure measuring device according to claim 1 wherein the blood pressure measuring device has a contact surface forming the second electrode.
